(19) United States
(12) Patent Application Publication
(10) Pub. No.: US 2012/0189544 A1
(43) Pub. Date: Jul. 26, 2012

(54) GASTROINTESTINAL DISEASE OR DISORDER IMAGING AND TREATMENT

(75) Inventors: Reuben J. Shaw, San Diego, CA (US); David Shackelford, La Jolla, CA (US); Debbie Vasquez, La Jolla, CA (US)

(73) Assignee: SALK INSTITUTE FOR BIOLOGICAL STUDIES, La Jolla, CA (US)

(21) Appl. No.: 13/376,688

(22) PCT Filed: Jun. 11, 2010
(86) PCT No.: PCT/US10/38380

§ 371 Date: Mar. 6, 2012

Related U.S. Application Data
(60) Provisional application No. 61/186,844, filed on Jun. 13, 2009.

(51) Int. Cl. A61K 51/04

(52) U.S. Cl. 424/1.73

(57) ABSTRACT

The disclosure provides methods for detection, prognosis and diagnosis of gastrointestinal polyps, cancer diseases and disorders.

### CROSS REFERENCE TO RELATED APPLICATIONS

This application claims priority under 35 U.S.C. §119 from Provisional Application Ser. No. 61/186,844, filed Jun. 13, 2009, the disclosure of which is incorporated herein by reference.

### STATEMENT AS TO FEDERALLY SPONSORED RESEARCH

The U.S. Government has certain rights in this invention pursuant to Grant No. P01 CA120964 awarded by the National Institutes of Health and Grant No. P50 CA128346 awarded by the National Cancer Institute.

### TECHNICAL FIELD

The disclosure relates to methods for diagnoses and prognoses of cell proliferative diseases and disorders.

### BACKGROUND

Peutz-Jeghers syndrome (PJS) is a familial cancer disorder due to inherited loss of function mutations in the LKB1/STK11 serine/threonine kinase. PJS patients develop gastrointestinal hamartomas with 100% penetrance often in the second decade of life, and demonstrate an increased predisposition towards the development of a number of additional malignancies. Amongst mitogenic signaling pathways, the mammalian-target of rapamycin complex 1 (mTORC1) pathway is uniquely hyperactivated in tissues and tumors derived from LKB1-deficient mice. Consistent with a central role for mTORC1 in these tumors, rapamycin as a single agent results in a dramatic suppression of pre-existing GI polyps in LKB1^{+/-} mice.

### SUMMARY

Peutz-Jeghers syndrome (PJS) is a familial cancer disorder due to inherited loss of function mutations in the LKB1/STK11 serine/threonine kinase. Loss of this tumor suppressor parallels other mutations including Pten, Nf1, and Tsc2. Mutations in these genes are responsible for the inherited cancer syndromes Cowden's disease, Neurofibromatosis Type I, and Tuberous Sclerosis Complex, all collectively referred to as Phakomatoses and all sharing overlapping clinical features including the development of hamartomas.

The disclosure provides a method for evaluating the likelihood that a subject is sensitive to an mTOR inhibitor in the treatment of a phakomatoses or hamartoma disease or disorder comprising measuring ¹⁸F-fludeoxyglucose (FDG) uptake in a sample or tissue of the subject, wherein an increased uptake of FDG compared to a normal control is indicative of a subject that is sensitive to an mTOR inhibitor. In one embodiment, the method can further include assessing the expression of or a mutation in a marker selected from the group consisting of PTen, Nf1, Tsc2, LKB1, mTOR, AMPK or any combination thereof. In one embodiment, the assessing comprises determining the expression level of a nucleic acid that encodes the PTen, Nf1, Tsc2, LKB1, mTOR, or AMPK protein. In another embodiment, the mTOR inhibitor is rapamycin or a rapamycin analog. In yet another embodiment, the rapamycin analog is selected from the group consisting of: temsirolimus (CCI-779), everolimus (RAD001; Certican), and AP23573. In one embodiment, a sample is collected from a subject and FDG analysis is performed on cells from the sample. In a further embodiment, the cells are obtained from the gastrointestinal system. In yet a further embodiment, the cells are from a polyp. In yet another embodiment, the phakomatoses disease or disorder is associated with a cell proliferative disorder selected from the group consisting of: colon cancer, breast cancer, or endometrial cancer. In one embodiment, the phakomatoses or hamartoma disease or disorder is selected from the group consisting of Cowden's disease, Neurofibromatosis Type I, and Tuberous Sclerosis Complex and Peutz-Jeghers syndrome. In yet another embodiment, an aberrant expression of mTOR or LKB1 and an increase in FDG uptake is indicative of a phakomatoses or hamartoma disease or disorder treatable with an mTOR inhibitor.

The disclosure also provides a method for evaluating the likelihood that a hamartomas disease or disorder is sensitive to an mTOR inhibitor comprising measuring ¹⁸F-fludeoxyglucose (FDG) uptake in a sample or tissue of the subject, wherein an increased uptake of FDG compared to a normal control is indicative of a subject that is sensitive to an mTOR inhibitor. In one embodiment, the uptake is measured by Fluorodeoxyglucose Positron emission tomography.

The disclosure also provides a method for evaluating the likelihood that a hamartomas disease or disorder is sensitive to an mTOR inhibitor comprising determining a mutation in a gene selected from the group consisting of PTen, Nf1, Tsc2, LKB1, mTOR, AMPK or any combination thereof and measuring glucose metabolism using Fluorodeoxyglucose Positron emission tomography.

The disclosure also provides a method of treating a subject with a hamartomas or phakomatoses disease or disorder, the method comprising administering an mTOR inhibitor to the subject, wherein the likelihood that the hamartomas or phakomatoses disease or disorder is sensitive to the mTOR inhibitor has been evaluated according to a method described above.

The disclosure also provides a method of determining an effective treatment with and mTOR inhibitor comprising carrying out the method described above, before and after administration of an mTOR inhibitor, wherein a decrease in FDG uptake is indicative or an effective treatment.

The disclosure also provides a method of detecting a Peutz-Jeghers syndrome comprising performing Fluorodeoxyglucose Positron emission tomography (FDG-PET).

The disclosure further provides a method of identifying a gastroinstestinal disease or disorder treatable with an mTOR inhibitor comprising performing Fluorodeoxyglucose Positron emission tomography (FDG-PET) and determining the presence of polyps.

The disclosure also provides a method of determining the prognosis or measuring the efficacy of a treatment for a hamartomas disease or disorder, an mTOR-dependent cell proliferative disorder, or an LKB-associated cell proliferative disease or disorder comprising performing Fluorodeoxyglucose Positron emission tomography (FDG-PET) before and after administration of a mTOR inhibitor.

The disclosure provides a method for determining the presence of a hamartomas disease or disorder, an mTOR-dependent or LKB-associated cell proliferative disorder comprising measuring uptake of FDG in a tissue or subject using Fluorodeoxyglucose Positron emission tomography.

The disclosure demonstrates the utility of the mTORC1 inhibitor rapamycin as a targeted therapeutic for the treatment of LKB1-deficient tumors, rapamycin as a single agent results in a dramatic suppression of pre-existing GI polyps in LKB1^{+/-} mice.

Furthermore, the disclosure demonstrates that these polyps, as well as LKB1- and AMPK-deficient murine embryonic fibroblasts, show dramatic up-regulation of the HIF-1α transcription factor and its downstream transcriptional targets in an mTORC1-dependent manner. The HIF-1α targets hexokinase II and Glut1 are upregulated in these polyps. Utilizing FDG-PET (Fluorodeoxyglucose Positron emission tomography) the disclosure demonstrates that LKB1^{-/+} mice show increased glucose utilization in focal regions of their GI tract corresponding to these gastrointestinal hamartomas. Though most often used in the detection of malignant tumors, these findings demonstrate that FDG-PET has clinical utility for the identification of polyps in PJS patients. Moreover, FDG-PET may be useful for monitoring the efficacy of treatment or surgical resection of these polyps.

Clinicians/oncologists can use FDG-PET imaging to localize and find all polyps within an individual, to help guide surgical resection as well as to monitor effect of chemotherapeutic treatment on tumor size. This would be applicable not only to patients with Peutz-Jeghers Syndrome but also to individuals with one of several other hamartoma syndromes (Cowden's disease, Neurofibromatosis Type I, and Tuberous Sclerosis Complex, Von-Hippel Lindau Syndrome).

FDG-PET may also therefore be useful in detecting sporadically arising tumors with mutations in the LKB1 gene, including the 10-30% of human lung cancers (NSCLC) showing mutations in this gene. The detection of a positive FDG-PET signal in human NSCLC may therefore dictate the therapeutic regiment chosen.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-F shows rapamycin reduces polyposis, mTORC1 signaling, and proliferation in Lkb1^{+/-} polyps (A) The top panel are images of whole stomach and duodenum and the bottom panel are images of the open stomachs (S) showing the exposed polyps (P) from Lkb1^{+/-} mice treated with either vehicle (VEH) (i,iv) or rapamycin (RAPA) (ii,v) and Lkb1^{+/+} mice treated with vehicle (VEH) (iii, vi). (B) Immunohistochemical analysis of polyps from VEH or RAPA treated Lkb1^{+/-} mice: H & E staining (i, ii), P-S6 staining (iii, iv), and Ki67 staining (v, vi). Results are representative of polyps from 5 mice of each treatment group. (C) A graph of the total polyp burden in Lkb1^{+/-} mice treated with either VEH (open circles, n=11) or RAPA (closed circles, n=10). The mean polyp burden for RAPA treated mice (2.0±1.2) was significantly reduced (*p value=0.00026; Student t test, 2 tail) compared to those mice treated with VEH (9.6±5.5). (D) Average polyp number in VEH treated mice (black bar, n=11) or RAPA treated mice (gray bar, n=10). Only visible polyps between 1 and 5 mm were scored in both VEH and RAPA treated mice. The mean polyp number for RAPA treated mice (2.8±1.4) was significantly reduced (*p value=0.00022; Student t test, 2 tail) compared to VEH treated mice (5.3±1.8). (E) Average polyp size in VEH treated mice (black bar, n=11) or RAPA treated mice (gray bar, n=10). The mean polyp size in RAPA mice (1.2±0.9) was significantly reduced (*p<0.0001 Student t test, 2 tail) compared to VEH treated mice (4.4±0.8). (F) Average percent of Ki67 positive epithelial cells in VEH treated mice (black bar, n=5) or RAPA treated mice (gray bar, n=5). The mean percent of Ki67 positive cells in RAPA mice (24.5±6.1) was significantly reduced (*p<0.0002 Student t test, 2 tail) compared to VEH treated mice (59.7±7.3).

FIG. 2A-C shows upregulated HIF-1α and HIF-1α targets in LKB1-deficient polyps and fibroblasts are reduced by rapamycin. (A) Immunoblots of lysates made from GI tissue or polyps from Lkb1^{+/+} and Lkb1^{+/-} mice treated VEH or RAPA. Immunoblots were probed against the indicated antibodies. (B) Immunohistochemical analysis of polyps from VEH or RAPA treated Lkb1^{+/-} mice probed with antibodies against Glut1 or Hif-1α. Results are representative of polyps from 3 mice of each treatment group. (C) Immunoblots of lysates from Lkb1^{+/+} or Lkb1^{-/- MEFs (left panel) or Ampk}^{+/+} or Ampk^{-/-} MEFs (right panel) probed with antibodies against the indicated proteins. MEFs were either untreated (NT) or treated with RAPA or cobalt chloride (CoCl₂).

FIG. 3A-B shows polyps from Lkb1^{+/-} mice visualized by FDG PET analysis. (A) Top left panel shows FDG PET images of axial, sagital and coronal views of untreated 12 month-old Lkb1^{+/+} mice. The top right panel shows the same views of untreated Lkb1^{+/-} mice. The FDG PET images of the mice are labeled accordingly, K=kidney, S=stomach, H=heart, B=bladder and P=polyp. (B) Bottom left panel shows FDG PET imaging of axial, sagittal and coronal views of Lkb1^{+/+} and Lkb1^{+/-} mice either treated with vehicle or rapamycin at 11 months of age. The bottom right panel shows the same mice imaged after one month of receiving either vehicle or rapamycin. The images of the mice are labeled accordingly, K=kidney, S=stomach, H=heart, B=bladder and P=polyp.

FIG. 4A-F shows polyps from human Peutz-Jeghers patients show increased P-S6, GLUT1 and HIF-1α expression. (A, B) The upper panels represent immunohistochemistry performed on human small bowel samples from normal patients (left) or Peutz Jeghers patients (right) that were probed with antibodies against the mTORC1 marker P-S6. (C-F) The middle and lower panels represent immunohistochemistry performed on normal colonic mucosa (left) and colonic Peutz-Jeghers polyps (right) probed with antibodies against the GLUT1 protein (C, D) or the HIF-1α protein (E, F).

FIG. 5A-B show rapamycin treatment of Lkb1^{+/-} mice reduces mTORC1 signaling. (A) Schematic showing the time line from which the mice were dosed with either VEH or RAPA. (B) Immunoblots of lysates of polyps or liver from Lkb1^{+/-} mice treated with VEH or RAPA. Immunoblots were probed with antibodies against the indicated proteins.

FIG. 6A-B shows polyps from Lkb1^{+/-} mice are normoxic while retaining HIF-1a expression Immunohistochemical (IHC) and immunocytochemical (ICC) analysis of gastrointestinal sections from 13-month old Lkb1^{+/-} and Lkb1^{+/+} mice probed with antibodies against Hif-1a or Hypoxyprobe-1. (A) Left panel represents IHC on polyps probed with an antibody against Hif-1a. The right panel represents ICC on polyps probed with an antibody against Hypoxyprobe-1 as indicated by positively stained cells in green. DAPI was used as a nuclear counter stain (blue). (B) IHC on gastrointestinal sections probed with an antibody against Hypoxyprobe-1. Arrows indicate positively stained epithelial cells in the small intestine. Dashed lines represent the separation between pyloric region and small intestine. IHC and ICC results are representative of polyps from 3 mice of each genotype.

### DETAILED DESCRIPTION

As used herein and in the appended claims, the singular forms “a,” “and,” and “the” include plural referents unless the context clearly dictates otherwise. Thus, for example, reference to “an inhibitor” includes a plurality of such inhibitors and reference to “the cell” includes reference to one or more cells, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

The disclosure demonstrates the use of fludeoxyglucose (FDG) positron imaging (FDG-PET) for detection of colorectal cancers and polyps, PJ disease and disorders, as well as various cell proliferative diseases and disorders comprising hamartomas.

Such methods can be used to diagnose colorectal cancers and polyps that would be responsive to rapamycin, or other kinase inhibitors, or which have LKB mutations and thus would be responsive to rapamycin.

The disclosure demonstrates that mutated LKB in various cancers induces increased glucose metabolism through, for example, upregulation of HIF-1-alpha and Glut6. Furthermore, that such upregulation and increase in glucose metabolism is present in various gastrointestinal disease and disorders including, but not limited to, PJ and related hamartomas.

Though most often used in the detection of malignant tumors, the disclosure provides that FDG-PET provides utility for the identification of polyps in PJS patients. Moreover, FDG-PET is useful for monitoring the efficacy of treatment or surgical resection of these polyps. In addition, secondary cancers that arise in PJS patients at other sites (breast, pancreas, endometrium) can also be visualized by FDG-PET, and whether rapamycin analogs or mTOR kinase inhibitors are useful therapies in the treatment of those tumors

Germline mutations in LKB1, TSC2, or PTEN tumor suppressor genes result in hamartomatous syndromes with shared tumor biological features. LKB1 is required for repression of mTOR under low ATP conditions in cultured cells in an AMPK- and TSC2-dependent manner, and that Lkb1 null murine embryonic fibroblasts (MEFs) and the hamartomatous gastrointestinal polyps from Lkb1 mutant mice show elevated signaling downstream of mTOR. These findings position aberrant mTOR activation at the nexus of these germline neoplastic conditions and suggest the use of mTOR inhibitors in the treatment of Peutz-Jeghers syndrome.

One aspect of successful treatment of various cell proliferative disorders including, but not limited to, cancers and neoplasms, is the identification of drug therapies for the specific biological cause of the cell proliferative disorders. For example, identification of cell proliferative disorders caused by kinases will assist in identifying those cell proliferative diseases and disorders that are likely to be responsive to a particular inhibitor drug. Various kinase inhibitors work by targeting a mutant kinase mutation having an effect on signaling molecules. These pathways can be modulated by loss of negative regulators leading to kinase activation.

As noted above, while researchers have identified a variety of genes and pathways involved in pathologies such as cancer, there is need in the art for additional tools to facilitate the analyses of the regulatory processes that are involved in disregulated cell growth. Moreover, an understanding of how the products of genes involved in disregulated cell growth interact in a larger context is needed for the development of improved diagnostic and therapeutic methods for identifying and treating pathological syndromes associated with growth disregulation. In particular, identifying signal transduction events driving oncogenesis provides therapeutic targets useful for assessing progression or inhibition of the oncogenic phenotype.

Aberrant activation of the mTORC1 pathway has been observed in spontaneously arising tumors in mice genetically engineered for loss of the tumor suppressors Pten, Nf1, Tsc2, or Lkb. Mutations in these genes are responsible for the inherited cancer syndromes Cowden's disease, Neurofibromatosis Type I, Tuberous Sclerosis Complex, and Peutz-Jeghers syndrome (PJS); collectively referred to as Phakomatoses and all sharing overlapping clinical features including the development of hamartomas. Biochemical and cell biological studies from the past decade have revealed that these tumor suppressors all are direct components of the mTOR signaling pathway that serve to inhibit mTORC1 activity.

Peutz-Jeghers syndrome (PJS) is an inherited autosomal dominant disorder in which patients develop benign hamartomatous polyps in the gastrointestinal tract and are predisposed to developing cancer. Inactivating mutations in the LKB1/STK11 tumor suppressor gene underlie PJS and have also been associated with sporadic lung adeno- and squamous carcinomas. Transgenic mice comprising homozygous deletion of Lkb1 is embryonic lethal to mice while heterozygous deletion of Lkb1 results in late onset gastrointestinal polyposis between 6-13 months of age that closely models human PJS. Gastrointestinal hamartomas are benign tumors that consist of hyperplastic glandular epithelial cells, disorganized tissue architecture and a characteristic arborizing smooth muscle stalk. Several hamartomatous syndromes involve inactivating mutations in genes that negatively regulate the mTORC1 pathway, which promotes cell growth and proliferation. In addition to Peutz-Jeghers Syndrome, these diseases include Cowden's disease, Tuberous Sclerosis Complex, and Neurofibromatosis Type I, due to inactivating mutation in the PTEN, TSC1, TSC2, or NF1 genes, respectively.

The LKB1 tumor suppressor is a serine/threonine kinase that is mutationally inactivated in the autosomal dominant Peutz-Jeghers syndrome (Boudeau et al., 2003), as well as in some sporadic lung adenocarcinomas (Sanchez-Cespedes et al. 2002; and Carretero et al. 2004). In the mouse, Lkb1 nullizygosity is embryonic lethal (at about embryonic day 9-10) due to vascular and neural tube defects (Ylikorkala et al., 2001), and Lkb1 heterozygosity engenders sporadic hamartomatous gastrointestinal polyps which are similar to those of PJS patients (Bardeesy et al. 2002; Miyoshi et al. 2002; Jishage et al. 2002; and Rossi et al. 2002). Hamartomas are benign tumors consisting of normal cellular differentiation but disorganized tissue architecture, and are present in several inherited tumor syndromes, including Cowden's disease/Bannayan-Zonana syndrome and tuberous sclerosis complex, which possess germline-inactivating mutations in the tumor suppressors PTEN and either TSC1 or TSC2, respectively.

The mammalian target of rapamycin (mTOR) is a central regulator of cell growth in all eukaryotes and is found in two functionally distinct multi-protein complexes. The mTOR complex 1 (mTORC1) is composed of mTOR and its scaffolding protein raptor. Signaling from mTORC1 is nutrient-sensitive, acutely inhibited by the bacterial macrolide rapamycin, and controls protein translation, cell growth, angiogenesis and metabolism. Activation of mTORC1 results in phosphorylation of a number of downstream targets involved in promoting cell growth and proliferation. These substrates include proteins involved in the regulation of protein translation such as the p70 S6 kinase 1 (S6K1) and eukaryotic initiation factor 4E-binding protein 1 (4EBP1). Amongst the mRNAs known to be translationally upregulated by mTORC1 are a number of key pro-growth proteins including cyclin D1, cyclin D3, Mcl-1, c-myc, and the hypoxia inducible factor 1 alpha (HIF-1α).

mTORC1 is activated by mitogenic stimuli acting through the PI3K/Akt and Erk signaling pathways. In contrast, under conditions of low intracellular ATP such as following nutrient deprivation or other stresses, the LKB1 tumor suppressor activates the AMP-activated protein kinase (AMPK), which then rapidly inhibits mTORC1 through phosphorylation of both raptor and the TSC2 tumor suppressor. Hence, treatment with AMPK activating drugs, or overexpression of LKB1 or AMPK, results in suppression of mTORC1, whereas targeted deletion of LKB1 in mice leads to increased mTORC1 activity in murine fibroblasts, liver, and in polyps of LKB1-heterozygous mice.

Despite the common feature of elevated mTORC1 signaling in hamartoma syndromes, the important targets of mTORC1 in LKB1-deficient tumors remain to be defined.

One of the earliest defined biochemical hallmarks of tumor cells is the propensity to rely on glycolysis for ATP production, even when oxygen is not limiting, unlike their normal counterparts. This conversion from oxidative phosphorylation to glycolysis that accompanies tumorigenesis was termed the Warburg Effect. In the past decade, interest in the Warburg effect has been renewed in part due to the increased use of ¹⁸F-Fluoro-deoxyglucose (FDG)-positron emission tomography (PET) in human cancer patients to detect tumors due to their higher rates of glucose utilization.

The disclosure shows that HIF-1α and its transcriptional targets in glucose metabolism are upregulated in LKB1-deficient tumors in mice and human Peutz-Jeghers patients. Increased Glut1 and Hexokinase II expression in these polyps of Lkb1^{+/-} mice allows them to be visualized by FDG-PET. As rapamycin strongly suppresses polyposis in the Lkb1^{+/-} mice, mTORC1 inhibitors and FDG-PET imaging are useful clinically in the treatment of PJS patients.

The disclosure demonstrates that polyps from PJ subjects, as well as LKB1- and AMPK-deficient mouse embryonic fibroblasts, show dramatic up-regulation of the HIF-1α transcription factor and its downstream transcriptional targets in a rapamycin-suppressible manner. HIF-1α targets, hexokinase II and Glut1, are upregulated in these polyps. Using FDG-PET cells comprising these mutations show increased glucose utilization in focal regions of their GI tract corresponding to gastrointestinal hamartomas. The disclosure demonstrates that polyps from human Peutz-Jeghers subjects similarly exhibit upregulated mTORC1 signaling, HIF-1α, and GLUT1 levels.

An underlying hypothesis is that mutational inactivation of tumor suppressors in individual cells lead to cell-autonomous hyperactivation of mTORC1, promoting cell growth and ultimately resulting in tumors that are subsequently reliant on mTORC1 signaling for tumor maintenance. Consistent with this possibility, rapamycin analogs have been examined for their therapeutic efficacy in the suppression of tumors that arise in a number of mouse models. The Pten^{+/-}, Nf1^{+/-}, Tsc^{+/-}, Lkb1^{+/-}, and activated Akt transgenic mouse models have also proven to be responsive to the mTOR inhibitors rapamycin or rapamycin analogs RAD001 (Novartis), CCI0779 (Wyeth) and AP23573 (Ariad). These drugs have been proven to effectively inhibit mTORC1 in vivo and reduce tumor burden through mTORC1 dependent mechanisms, including suppression of cyclin D, Mcl-1, or HIF-1α and its targets.

In recent clinical trials, rapamycin and its analog Temsirolimus were shown to have palliative success in clinical trials on patients with PTEN-deficient glioblastomas and metastatic renal cell carcinomas. Furthermore, in a pair of phase II clinical trials involving tuberous sclerosis (TSC) and lymphangioleiomyomatosis (LAM) patients, partial responses to the rapamycin analog Sirolimus were observed, including regression of angiomyoliomas with continuous therapy, consistent with previous clinical observations in TSC patients given rapamycin. Combined with data from mouse models, these clinical data suggest that hamartoma syndromes with hyperactivation of mTORC1 may be particularly responsive to rapamycin analogs as a single agent. To date there are no therapies to treat PJS and the only course of treatment is resection of arising gastrointestinal hamartomatous polyps. As mentioned above, the disclosure demonstrates that rapamycin greatly reduced the polyp burden in the Lkb1^{+/-} mouse model of PJS. This suppression was correlated with inhibition of mTORC1 and downregulation of HIF-1α and its transcriptional targets. While these results are encouraging for the use of rapamycin analogs as therapeutics for PJS, like the recent Phase II clinical trial findings with TSC patients, removal of the drug may result in rapid return of the initial tumor due to the largely cytostatic nature of the response. Perhaps new, targeted inhibitors directed at the kinase domain of mTOR will produce greater therapeutic response with targeted cytotoxicity, or perhaps kinase inhibitors that dually inactivate mTOR and PI3K, as PI3K provides a survival signal in most epithelial cell types. As observed in most cancers studied to date, combinations of targeted therapeutics, or of targeted and traditional chemo-therapeutics may find the ultimate utility in the treatment of this disease. Importantly, it is worth noting here that rapamycin treatment may not only be therapeutically useful for the hamartomas that arise in Peutz-Jeghers patients, but also in preventing and reducing any secondary malignancies that arise in these patients at additional sites (breast, pancreas, ovary).

The disclosure also demonstrates the transcription factor HIF-1α as a relevant target of mTORC1 in LKB1-dependent hamartomas, and the upregulation of HIF-1α targets Glut1 and Hexokinase II are responsible for the ability of these tumors to be visualized by FDG-PET. HIF-1α has previously been shown to be an excellent correlate of rapamycin response in a transgenic model of prostate neoplasia dependent on activated Akt, as well as in VHL-deficient renal cell carcinoma xenografts. Consistent with the findings here in spontaneously arising hamartomas in LKB1^{-/+} mice, recent studies using human glioblastoma xenografts and transplanted murine breast carcinomas also found rapamycin-sensitivity of FDG-PET imaging of these tumors. Data from LKB1 and AMPK-deficient MEFs demonstrate that HIF-1α and HIF-1α targets are dramatically upregulated in these cells under normoxic conditions, indicating that HIF-1α upregulation is not a secondary consequence of other tumor mutations—or hypoxia—present within the hamartomas. These findings also suggest that AMPK may be a key effector of LKB1 in the suppression of mTORC1 and HIF-1α in the normal gastrointestinal epithelium that when disrupted gives rise to hamartomas. Interestingly, the increase in HIF-1α was observed in the deficient fibroblasts under conditions of increased cell density when basal AMPK activity is high in the wild-type cells, perhaps due to glucose depletion of the media. In cells that genetically lack the ability to activate AMPK, HIF-1α is upregulated in an mTORC1-dependent manner under these conditions.

Though most often used in the detection of malignant tumors, the disclosure provides that FDG-PET may find clinical utility for the identification of polyps in PJS patients. Moreover, FDG-PET may be useful for monitoring the efficacy of treatment or surgical resection of these polyps. In addition, secondary cancers that arise in PJS patients at other sites (breast, pancreas, endometrium) can also be visualized by FDG-PET, and whether rapamycin analogs or mTOR kinase inhibitors are useful therapies in the treatment of those tumors.

Furthermore, like HIF-1α and its target genes, the FDG-PET signal in the GI tract of these mice is abolished by rapamycin treatment. These findings suggest a number of therapeutic modalities for the treatment and detection of hamartomas in PJS patients and for the screening and treatment of the 30% of sporadic human lung cancers, as well as other cell proliferative diseases and disorders, bearing LKB1 mutations.

A cell proliferative disease or disorder refers generally to cells that have an aberrant growth compared to normal cells. Examples of cells comprising a cell proliferative disease or disorder include neoplastic cells and cancer cells. The terms “cancer”, “cancerous”, or “malignant” refer to or describe a disease or disorder characterized by unregulated cell growth. Examples of cancer include but are not limited to astrocytoma, blastoma, carcinoma, glioblastoma, leukemia, lymphoma and sarcoma. More particular examples of such cancers include adrenal, and ophthalmologic cancers, brain cancer breast cancer, ovarian cancer, colon cancer, colotectal cancer, rectal cancer, squamous cell cancer, small-cell lung cancer, non-small cell lung cancer, Hodgkin's and non-Hodgkin's lymphoma, testicular cancer, esophageal cancer, gastrointestinal cancer, renal cancer, pancreatic cancer, glioblastoma, cervical cancer, glioma, liver cancer, bladder cancer, hepatoma, endometrial carcinoma, salivary gland carcinoma, kidney cancer, liver cancer, prostate cancer, vulval cancer, thyroid cancer, hepatic carcinoma and various types of head and neck cancer.

Fludeoxyglucose (FDG) or Fluorodeoxyglucose is a glucose analog. Its full chemical name is 2-fluoro-2-deoxy-D-glucose, commonly abbreviated to FDG. FDG is most commonly used in the medical imaging modality positron emission tomography (PET). The fluorine in the FDG molecule is chosen to be the positron-emitting radioactive isotope fluorine-18, to produce ¹⁸F-FDG. After FDG is delivered to a subject, a positron emission tomography scanner can form images of the distribution of FDG around the body. The images can be assessed by a nuclear medicine physician or radiologist to provide diagnoses of various medical conditions.

FDG is taken up by high-glucose-using cells where phosphorylation prevents the glucose from being released intact. The 2-oxygen in glucose is needed for further glycolysis, so that (in common with 2-deoxy-D-glucose) FDG cannot be further metabolized in cells, and therefore the FDG-6-phosphate formed does not undergo glycolysis before radioactive decay. As a result, the distribution of ¹⁸F-FDG is a good reflection of the distribution of glucose uptake and phosphorylation by cells in the body.

In PET imaging, ¹⁸F-FDG can be used for the assessment of glucose metabolism hamartomas, polyps and cancer lesions of, for example, the lungs and gastrointestinal tract. As described herein ¹⁸F-FDG is taken up more actively by cells having metabolic changes characteristic of cell proliferative disorders, wherein the FDG is phosphorylated by hexokinase (whose mitochondrial form is greatly elevated in rapidly-growing malignant tumours), and retained by tissues with high metabolic activity, such as most types of malignant tumours. As a result FDG-PET can be used for diagnosis, staging, and monitoring treatment of cancers,

In body-scanning applications in searching for tumor or metastatic disease, a dose of FDG in solution (typically 5 to 10 millicuries or 200 to 400 MBq) is typically injected rapidly into a saline drip running into a vein, in a subject who has been fasting for at least 6 hours, and who has a suitably low blood sugar. The patient must then wait about an hour for the sugar to distribute and be taken up into organs. The subject is then placed in the PET scanner for a series of one or more scans which may take from 20 minutes to an hour.

An FDG signal is higher in subjects with mutations in LKB thus demonstrating the utility of these modes of bioimaging to select the appropriate subject who will benefit most from molecularly targeted therapies comprising rapamycin and other kinase inhibitors that can modulate the activity of glucose metabolism through kinase regulated mechanisms. In addition, repeated FDG-PET imaging can be used to measure whether a treatment is effective. For example, a treatment may be provided to a subject and one or more FDG-PET images performed to determine any change in the metastasis, spread, growth or size of a polyp or cancer lesion.

The following examples are intended to illustrate but not limit the disclosure. While they are typical of those that might be used, other procedures known to those skilled in the art may alternatively be used.

### EXAMPLES

For immunoblotting, anti-phospho-S6K1 (T389), phospho-ribosomal protein S6 (S235/236), elF4E, phospho-4-ebp1 (Ser65), hexokinase II (C64G5), phospho-Akt (Thr308) and bNIP3 antibodies were obtained from Cell Signaling Technology (Beverly, Mass.). Antibodies against HIF-1α (C-term) polyclonal antibody (Cayman Chemicals Ann Arbor Mich.), Glut1 (Alpha Diagnostics Int., TX, USA; GT11-A Rabbit polyclonal antibody), Cyclin D1 (BD Pharmingen, San Diego, Calif.) and tubulin (Sigma Chemicals) were also used. Rapamycin was obtained from LC laboratories (Woburn, Mass.).

Cell Culture. All cells were incubated at 37° C. maintained at 5% CO₂. Littermate derived Lkb1^{+/+} and Lkb1^{-/-} MEFs were isolated from day 14 post-coitum Lkb1^{+/+} and Lkb1^{lox/lox} embryos and grown in DMEM medium plus 10% fetal bovine serum (Hyclone), penicillin and streptomycin. Both genotypes were infected with a Cre expressing adenovirus and subsequently immortalized with an INK4a shRNA expressing lentivirus. Ampkα1/α2^{+/+} and Ampkα1^{-/-} MEFs were plated at a density of 1×10⁵ and Lkb1^{+/+} and Lkb1^{-/-} MEFs were plated 2.0×10⁵ per well in 6 well dishes and grown in DMEM medium plus 10% fetal bovine serum (Hyclone), penicillin and streptomycin. 24 hours after plating, MEFs were then left untreated, treated with 50 nM rapamycin (LC laboratories) or 100 μM CoCl₂ (Sigma Aldrich) for 24 hours.

Mouse Colony Maintenance, Treatment Regimen and Polyp Measurement. Lkb1^{+/+} and Lkb1^{+/-} mice, maintained on an FVB/N genetic background, were monitored for the development of gastrointestinal polyps. Mice with clinical signs of disease were euthanized and necropsied. The groups were vehicle treated or RAPA treated mice. Mice were treated with either vehicle (5% Tween 80, 5% PEG400 solution), or 10 mg/kg rapamycin by intraperitoneal injection once a day for 5 days with 2 days rest for a period of either 1 or 2 months as indicated. The mean latency, distribution of polyps, and polyp phenotype were comparable to previous studies. Polyps were scored and total polyp burden was measured). Ki67 stained polyps were scored from 5 polyps for each group (Vehicle or Rapamycin treated). For each polyp, three fields of view at a 32× magnification were randomly selected and scored. A total of 200 nuclei from epithelial cells total per field of view were counted. Within the same field all Ki67 positively stained nuclei from epithelial cells were counted. The number of Ki67⁺ cells were divided by the total number of nuclei and the percentages for each group were averaged and a p-value was determined.

Tissue Isolation and Biochemistry. Polyps and adjacent tissue were harvested immediately and either processed for histological analysis or snap frozen in liquid nitrogen for molecular studies. These samples were then placed frozen into Nunc tubes and homogenized in lysis buffer (20 mM Tris [pH 7.5], 150 mM NaCl, 1 mM EDTA, 1 mM EGTA, 1% Triton X-100, 2.5 mM pyrophosphate, 50 mM NaF, 5 mM β-glycerophosphate, 50 nM calyculin A, 1 mM Na3VO4, Complete protease inhibitor cocktail (Roche Diagnostics, Indianapolis Ind.) on ice for 30 sec using a tissue homogenizer. MEFs were lysed in boiling SDS-lysis buffer (10 mM Tris [pH 7.5], 100 mM NaCl, 1% SDS) after the indicated treatments. After trituration, lysates were equilibrated for protein levels using the BCA method (Pierce, Rockford, Ill.) and resolved on 6%-12% SDSPAGE gels, depending on the experiment. Gels were transferred to PVDF and Western blotted according to the antibody manufacturer's suggestions.

Histology and Immunohistochemistry. Mouse tissues were fixed in 10% formalin overnight and embedded in paraffin. For immunohistochemistry, slides were deparaffinized in xylene and ethanol and rehydrated in water. Antigen retrieval using sodium citrate pH 6.0 or Tris EDTA pH 10.0 buffer was performed according to the manufacturer's instructions. Slides were quenched in hydrogen peroxide (3%) to block endogenous peroxidase activity and then washed in TBST buffer. Slides were blocked in 5% normal serum for 1 hr at room temperature. Slides were incubated with primary antibody diluted in blocking buffer, washed and a secondary biotinylated goat-anti mouse IgG antibody was applied. The avidin-biotin peroxidase complex method (Vector, Birlingame, Calif.) was used and staining was visualized using the DAB chromophore (Vector ABC; DAB). Slides were counterstained with hematoxylin and mounted with Fluoromount (SoutherBiotech, Birmingham, Ala.). The anti-phospho ribosomal protein S6 (S235/236) (Cell Signaling Technology, Beverly, Mass.), Ki67 (SP6) (Neomarkers, Fremont, Calif.) and GLUT1 (Alpha Diagnostics Int., TX, USA; GT11-A Rabbit polyclonal antibody) and HIF-1α (Novus Biologicals LLC., Littleton, Colo., USA; Rabbit polyclonal antibody) antibodies were diluted according to manufacturer's suggestions. For immunochemistry on human PJS and control samples, the avidin-biotin complex (ABC) method was performed. Briefly, slides were deparaffinized and endogenous peroxidase activity was blocked by incubation in 3% H₂O₂ for 10 min at room temperature. For antigen retrieval, sections were heated in pressure cooker in 10 mM citrate buffer (pH 6.0) for pS6 (Ser235/236; Cell Signaling Technology, MA, USA; Rabbit polyclonal antibody; 1:200 dilution), and in Tris-EDTA buffer (pH 8.0) for GLUT1 (Alpha Diagnostics, GT12-A 1:100 dilution) before staining. The P-S6 and GLUT1 antibodies were applied and incubated overnight at 4° C. Application of the monoclonal antibody for HIF-1alpha (clone H1α67; Cat#NB100-105; Novus Biologicals, CO, USA; Mouse monoclonal antibody; 1:10,000 dilution) was followed by sequential 15 minutes incubations with biotinylated link antibody, streptavidin-biotin-peroxidase complex, amplification reagent and streptavidin-peroxidase. Immunodetection was performed with an LSAB 2 system (DAKO; Carpinteria, Calif.). Hematoxylin was used as a counterstain. A known GLUT1 and P-S6 positive breast carcinoma or normal skin sample with known HIF-1α expression were used as a positive controls and the same samples were used as negative controls by replacing the primary antibody with PBS. For every sample, immunostaining was repeated at least twice to confirm the results. Staining was graded for intensity as negative or positive by two independent observers (CLW and SW) and there were no major discrepancies between the two observers.

Immunocytochemistry and Imaging. For immunocytochemistry, sections were deparaffinized in xylene and ethanol and rehydrated in water. Antigen retrieval was performed using sodium citrate pH 6.0. Sections were blocked in 5% normal serum for 1 hr at room temperature and incubated with Hydroxyprobe-1 mouse monoclonal antibody 1 (MAb1; Natural Pharmacia International Inc) at a 1:50 dilution for 40 min at RT. A secondary anti-rabbit Alexa488 secondary antibody (Molecular Probes, 1:1000) and DAPI stain were then used. Coverslips were mounted in FluoromountG (SouthernBiothech) and images were acquired on a Zeiss Axioplan2 epifluorescence microscope coupled to the Openlab software using the 20× objective.

Detection of Hypoxic Regions in Polyps. To allow assessment of the hypoxic regions within tumors, mice were injected i.p. with 60 mg/kg (in w/v PBS) pimonidazole (Hydroxyprobe-1™, Natural Pharmacia International Inc.), 1.5 hr prior to sacrifice. Stomach and small intestine were dissected out, processed, and embedded into paraffin. For immunohistochemistry, 5 μM sections were deparaffinized in xylene and ethanol and rehydrated in water. Antigen retrieval using sodium citrate pH 6.0 buffer was performed according to the manufacturer's instructions. Sections were quenched in hydrogen peroxide (3%) and then washed in TBST buffer. Sections were blocked in 5% normal serum for 1 hr at room temperature. And then incubated with Hydroxyprobe-1 mouse monoclonal antibody 1 (MAb1; Natural Pharmacia International Inc) at a 1:50 dilution for 40 min at RT. A secondary biotinylated goat-anti mouse IgG antibody was applied, and staining was visualized using the DAB chromophore (Vector ABC; DAB). Sections were counterstained with hematoxylin and mounted with Fluoromount (SoutherBiotech, Birmingham, Ala.).

Patient Samples. Formalin-fixed, paraffin-embedded sections of 5 PJPs located in small bowel and 8 PJPs located in colon were obtained from 8 patients with PJS (5 from Massachusetts General Hospital, USA, and 3 from Biomedicum Helsinki, Finland) for immunohistochemical staining with the P-S6 and GLUT1 antibodies. An additional 9 sections of formalin-fixed, paraffin-embedded PJPs (4 from colon, 4 from small intestine, 1 from stomach) were obtained from 7 patients with PJS from Massachusetts General Hospital, USA for immunohistochemical staining with the HIF-1α antibody. Normal small bowel mucosa controls and normal colonic mucosa controls were obtained at Massachusetts General Hospital. The studies were approved by the Human Study Committees in both hospitals. The histopathological diagnosis of PJPs was confirmed in all cases by two independent pathologists. Consecutive 5 μm tissue sections were cut from each tissue block for immunohistochemical analysis.

FDG PET Analysis. Mice were warmed for 30 minutes in an isolator box on a recirculating water pad kept at ˜30° C. in order to normalize their body temperature for injection (i.v. 250 uCi, 0.1 ml) of 18-fluoro-deoxyglucose (F18-FDG). Mice were anesthetized with 2% Isoflurane and kept under anesthesia until imaging (2 bed positions, 10 min per position) one hour after injection by microPET (Vista DR, GE Healthcare). Individual animals were scanned once or twice in a three week period. Ten scans were performed on +/− mice and 5 scans on +/+ mice.

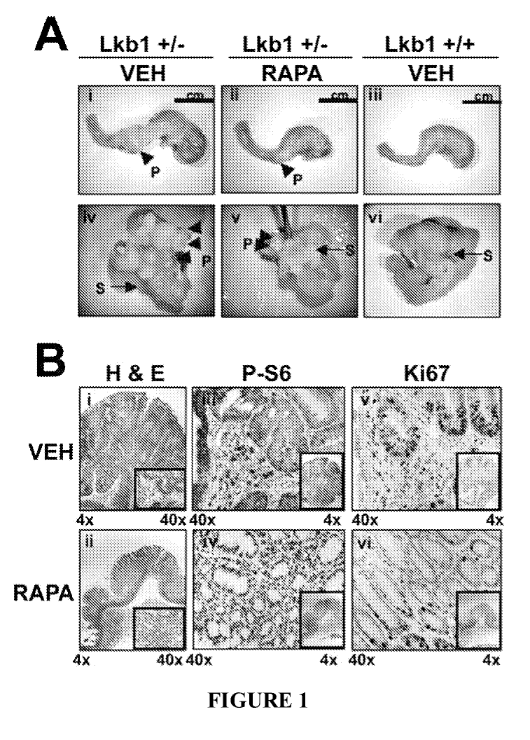

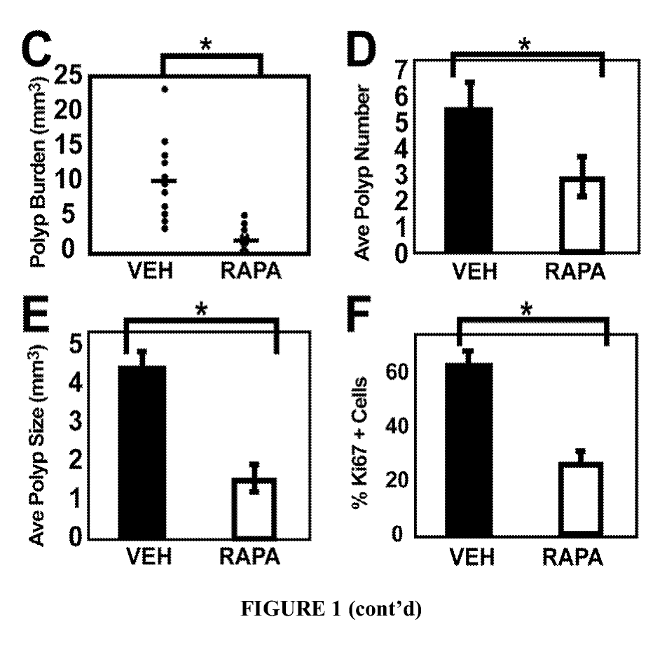

Rapamycin reduces tumor burden and proliferation in Lkb1^{+/-} mice. The effect of rapamycin on pre-existing PJS-like polyps was investigated, by treating 9 month old Lkb1^{+/-} or Lkb1^{+/+} mice for a period of two months with rapamycin or vehicle. The studies revealed that at 9 months of age, 100% of the Lkb1^{+/-} mice have developed multiple gastrointestinal hamartomas. Both Lkb1^{+/+} and Lkb1^{+/-} mice tolerated rapamycin treatment with no obvious cytotoxicity or immunosuppression at the doses utilized. After two months of treatment, polyp size and number in each mouse were quantitated. The wild-type Lkb1^{+/+} mice were free of polyps while all of the Lkb1^{+/-} mice treated with vehicle presented severe polyp burden at or before 11-12 months of age, consistent with previous studies of these mice. These mice had multiple large polyps in the stomach and pylorus and suffered from severe distention of the stomach and anemia (FIG. 1A panels i, iv). Histological analysis of H&E stained polyps from untreated mice were classified as pedunculated, hyperplastic lesions consisting of differentiated glandular epithelium, stroma, and a smooth muscle stalk. In contrast, Lkb1^{+/-} mice treated with rapamycin had a dramatic reduction in polyp burden. These mice uniformly had reduced polyp size (FIG. 1E) as well as lower frequency of polyps (FIG. 1D), no distention of the stomach (FIG. 1A panels ii, v) and appeared active and vigorous at 2 months of treatment. Comparison of the polyp burden between Lkb1^{+/-} mice treated or untreated with rapamycin showed an 80% reduction in the overall mass of polyps (FIG. 1C). Polyps isolated from treated mice were greatly reduced in size, though still retained some disruption of the normal tissue architecture as shown by H&E staining (FIG. 1B panel ii).

mTORC1 signaling was analyzed in the polyps of Lkb1^{+/-} mice to determine whether rapamycin was effectively inhibiting the pathway. Immunohistochemical staining of polyps for phospho-S6 (P-S6) revealed that untreated polyps displayed high levels of P-S6 staining indicative of hyperactive mTOR signaling, while the polyps from rapamycin treated mice were greatly reduced for P-S6 staining indicating successful inhibition of mTORC1 signaling (FIG. 1B panels iii, iv). These results were further corroborated by western blot analysis of polyp lysates.

Rapamycin has been shown to suppress tumor growth and induce apoptosis, resulting in cytostatic or cytotoxic responses in several genetically engineered mouse models with spontaneously arising tumors. The disclosure analyzes the mechanism(s) by which rapamycin reduced polyp burden in the Lkb1^{+/-} mice. Expression of the proliferation marker Ki67 was analyzed in rapamycin or vehicle-treated polyps. The highest expression of Ki67 was found in proliferating epithelial cells at the base of the crypts. While staining was extensive in the vehicle-treated polyps, rapamycin-treated polyps showed a clear reduction in Ki67 staining (FIG. 1B panels v,vi; FIG. 1F). However, polyps from treated mice did not show evidence of apoptosis as detected by cleaved caspase-3 protein utilizing immunohistochemistry or immunoblotting. These results suggest that in this tumor model rapamycin may be having a cytostatic effect rather than a cytotoxic effect, consistent with observations in other tumor types.

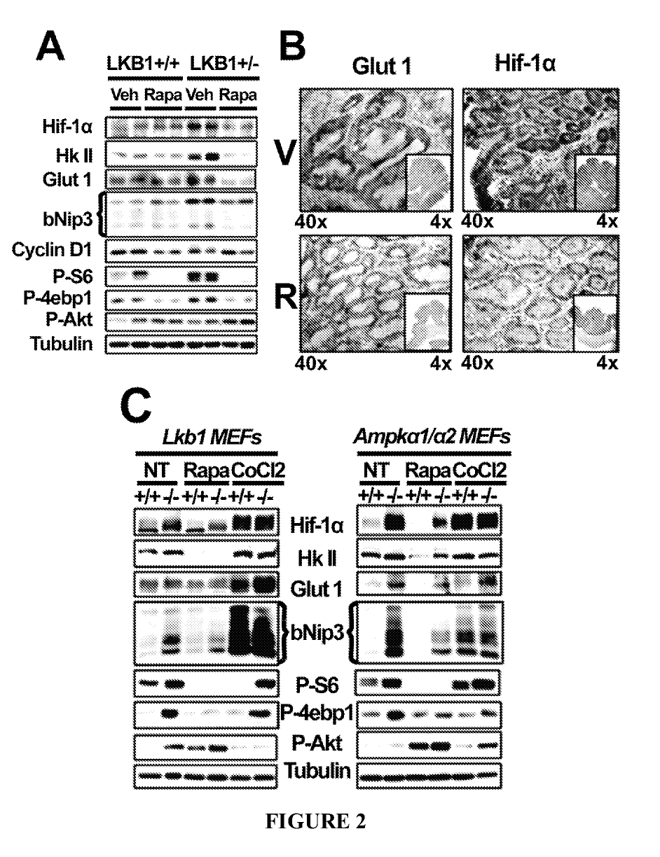

Rapamycin downregulates expression HIF-1α and HIF-1α targets. Activation of mTORC1 results in increased translation of a number of key downstream targets, including cyclin D1 and the hypoxia inducible factor 1 alpha gene (HIF-1α). The protein levels of cyclin D1 and HIF-1α were examined in the stomachs and polyps of Lkb1^{+/+} and Lkb1^{+/-} mice by western blot. HIF-1α but not cyclin D1 levels were elevated in polyps of vehicle-treated Lkb1^{+/-} mice. In contrast, the polyps of mice treated with rapamycin showed reduced HIF-1α levels similar to the basal levels seen in Lkb1^{+/+} mice (FIG. 2A). Next, protein expression of transcriptional targets of HIF-1α including Glut1, Hexokinase II, and bNIP3 were analyzed and observed that their expression was up in the polyps proportional to HIF-1α upregulation and similarly, that the expression of these HIF-1α targets was suppressed by rapamycin (FIG. 2A). To confirm that HIF-1α and its targets were upregulated within the epithelial cell population that are P-S6 and Ki67 positive, as opposed to originating from any stromal or infiltrating cells, immunohistochemistry was performed with anti-HIF-1α and Glut1 antibodies in vehicle- and rapamycin-treated LKB1^{+/-} polyps. HIF-1α and Glut1 protein expression were much higher in epithelial cells in the untreated polyps and was diminished with rapamycin treatment (FIG. 2B).

In order to determine if increased HIF-1α levels were due to loss of the Lkb1 gene and not simply a consequence of hypoxia within the polyp microenvironment or a secondary mutation that arose during polyp formation, the functional levels of hypoxia present in the polyps and surrounding epithelium using hypoxyprobe-1 were analyzed. No significant levels of hypoxia were observed in the polyps, in contrast to widespread HIF-1α elevation throughout the epithelial cells of the polyps. To further extend this analysis in a controlled normoxic environment, and to rule out the potential impact of any secondary mutations that may have arisen in the polyps which might contribute to upregulation of HIF-1α, HIF-1α levels were examined in primary non-immortalized wild-type and Lkb1-deficient MEFs grown in normoxic conditions. Expression of HIF-1α protein, as well as the HIF-1α targets Hexokinase II and bNIP3, were dramatically increased in Lkb1^{-/-} MEFs and all downregulated by rapamycin treatment (FIG. 2C). As previously shown that AMPK is a key target of LKB1 that controls mTORC1 activity via its phosphorylation of TSC2 and raptor, it was then examined whether HIF-1α and its targets were similarly upregulated in immortalized MEFs lacking both catalytic isoforms of AMPK. Indeed, HIF-1α and its targets were upregulated in Ampkα1/α2^{-/-} fibroblasts compared to wild type cells and treatment with rapamycin reduced their expression, paralleling suppression of 4ebp1 and S6K phosphorylation (FIG. 2C).

Lkb1^{+/-} polyps exhibit dramatic increases in glucose metabolism. One of the earliest defined biochemical hallmarks of tumor cells is the propensity to rely on glycolysis for ATP production, even when oxygen is not limiting, unlike their normal counterparts. This conversion from oxidative phosphorylation to glycolysis that accompanies tumorigenesis was termed the Warburg Effect. In the past decade, interest in the Warburg effect has been renewed in part due to the increased use of ¹⁸F-Fluoro-deoxyglucose (FDG)-positron emission tomography (PET) in human cancer patients to detect tumors due to their higher rates of glucose utilization. The molecular underpinning for increased FDG-PET has been hypothesized to involve increased levels of cell surface glucose transporters including GLUT1, as well the enzyme for the first committed step of glycolysis, hexokinase II. As immunoblotting had revealed increased expression of both GLUT1 and Hexokinase II in the polyps of LKB1^{+/-} mice, an interest was whether these tumors could be visualized by FDG-PET.

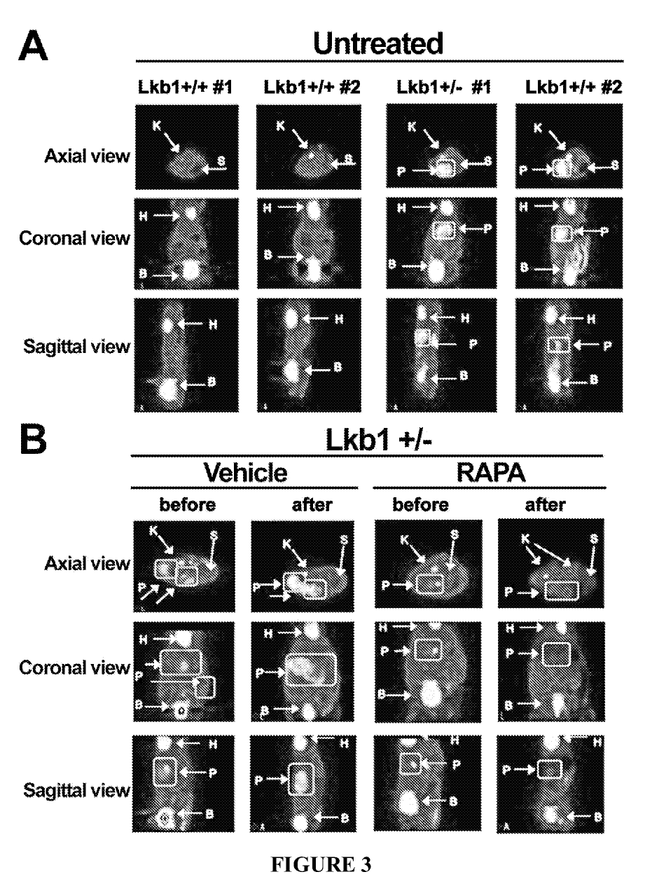

11 month old Lkb1^{+/+} and Lkb1^{+/-} mice were analyzed by FDG PET to scan for the presence of GI polyps. In addition to the excretion to the bladder, uptake of FDG in the heart and kidney of all mice regardless of genotype was analyzed. However, FDG PET images showed increased FDG in focal masses located in the Lkb1^{+/-} mice in their midline below the heart where the stomach and pylorus are located while the Lkb1^{+/+} were negative (P=0.06) for FDG signal in this area (FIG. 3A). Several of the Lkb1^{+/-} mice were sacrificed after imaging and it was confirmed that these animals had large polyps in the pylorus and stomach corresponding exactly to the regions of greatest FDG uptake (data not shown). Treatment of animals with rapamycin for 4 weeks abolished the FDG-PET signal. Immediate autopsy of the animals imaged by FDG-PET revealed that the rapamycin-treated mice had minimal detectable GI polyps while the vehicle-treated mice all exhibited the presence of large GI polyps (FIG. 3B). These results demonstrate that FDG-PET analysis is a viable method by which to detect polyps in Lkb1^{+/-} animals and confirms that rapamycin reverses polyp growth in Lkb1^{+/-} mice.

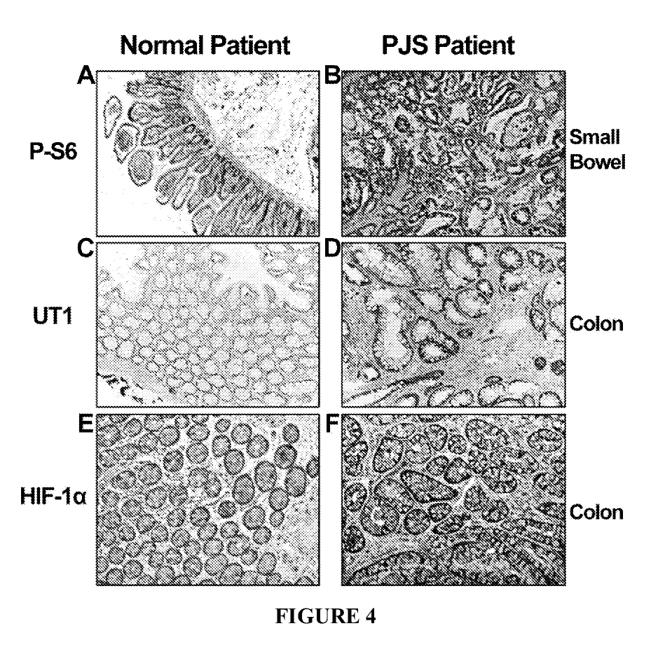

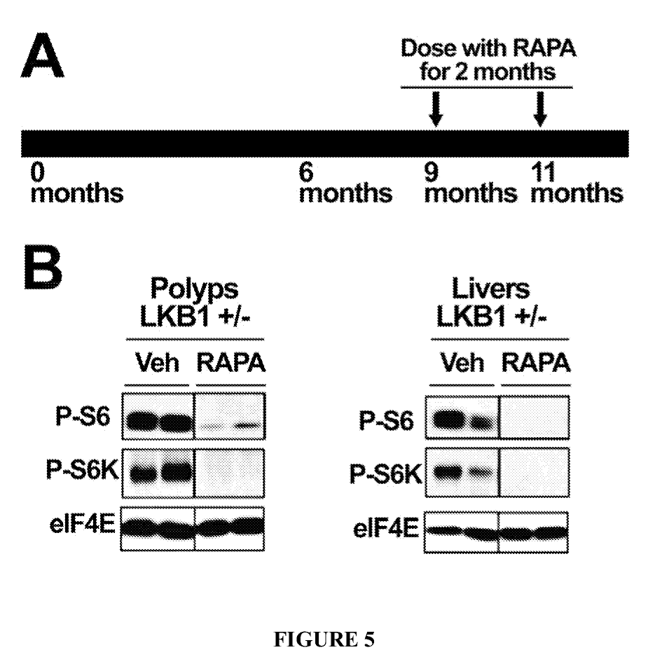

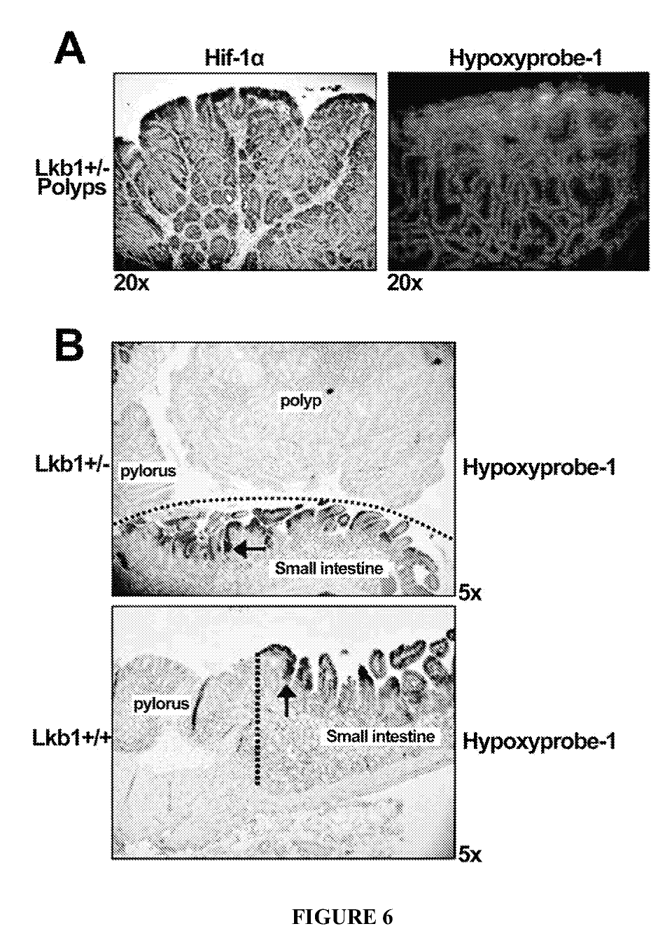

mTORC1 and HIF-1α signaling increased in human PJS polyps. The increased mTORC1 and HIF-1α dependent signaling observed in the LKB1^{+/-} murine model are relevant to human Peutz-Jeghers patients. mTORC1 signaling, HIF-1α protein, and GLUT1 protein expression were analyzed by immunohistochemistry in small bowel and colon samples from PJS patients and compared to samples of small bowel and colonic mucosa from normal patient controls. Expression of the mTOR target P-S6 was increased in the epithelium of small bowel of PJS patients compared to that of normal tissue (FIG. 4A,B). Likewise, strong immunostaining of HIF-1α and GLUT1 was observed in glandular epithelial cells in 7 of 8 PJP colonic polyp specimens (FIG. 4D,F). In the normal colonic mucosa specimens, weak immunohistochemical staining of HIF-1α and Glut1 was observed relative to the highly elevated levels observed in the PJS samples (FIG. 4C,E). These results suggest that loss of the LKB1 gene leads to both mTORC1 hyperactivation and increased GLUT1 expression in PJS patients in a manner that closely follows the murine model of PJS.

A number of embodiments of the disclosure have been described. Nevertheless, it will be understood that various modifications may be made without departing from the spirit and scope of the disclosure. Accordingly, other embodiments are within the scope of the following claims.

1. A method for evaluating whether a subject is sensitive to an mTOR inhibitor in the treatment of a phakomatoses or hamartoma disease or disorder comprising measuring ¹⁸F-fludeoxyglucose (FDG) uptake in a sample or tissue of the subject, wherein an increased uptake of FDG compared to a normal control is indicative of a subject that is sensitive to an mTOR inhibitor.

2. The method of claim 1, further comprising assessing the expression of a marker selected from the group consisting of PTen, Nf1, Tsc2, LKB1, mTOR, AMPK or any combination thereof.

3. The method of claim 1, wherein the mTOR inhibitor is rapamycin or a rapamycin analog.

4. The method of claim 1, wherein the rapamycin analog is selected from the group consisting of: temsirolimus (CCI-779), everolimus (RAD001; Certican), and AP23573.

5. The method of claim 1, wherein the sample comprises cells obtained from the subject.

6. The method of claim 5, wherein the cells are obtained from the gastrointestinal system.

7. The method of claim 6, wherein the cells are from a polyp.

8. The method of claim 2, wherein the assessing comprises determining the expression level of a nucleic acid that encodes the PTen, Nf1, Tsc2, LKB1, mTOR, or AMPK protein.

9. The method of claim 8, wherein the expression level of the marker is indicative of a mutation in the marker.

10. The method of claim 1, wherein the phakomatoses disease or disorder is associated with a cell proliferative disorder selected from the group consisting of: colon cancer, breast cancer, or endometrial cancer.

11. The method of claim 1, wherein the phakomatoses or hamartoma disease or disorder is selected from the group consisting of Cowden's disease, Neurofibromatosis Type I, and Tuberous Sclerosis Complex and Peutz-Jeghers syndrome.

12. The method of claim 2, wherein an aberrant expression of mTOR or LKB1 and an increase in FDG uptake is indicative of a phakomatoses or hamartoma disease or disorder treatable with an mTOR inhibitor.

13. A method for evaluating whether a hamartomas disease or disorder is sensitive to an mTOR inhibitor comprising measuring ¹⁸F-fludeoxyglucose (FDG) uptake in a sample or tissue of the subject, wherein an increased uptake of FDG compared to a normal control is indicative of a subject that is sensitive to an mTOR inhibitor.

14. The method of claim 1, wherein the uptake is measured by Fluorodeoxyglucose Positron emission tomography.

15-19. (canceled)

20. A method of detecting a Peutz-Jeghers syndrome comprising performing Fluorodeoxyglucose Positron emission tomography (FDG-PET).

21. A method of identifying a gastroinstestinal disease or disorder treatable with an mTOR inhibitor comprising performing Fluorodeoxyglucose Positron emission tomography (FDG-PET) and determining the presence of polyps.

22. (canceled)

23. A method for determining the presence of a hamartomas disease or disorder, an mTOR-dependent or LKB-associated cell proliferative disorder comprising measuring uptake of FDG in a tissue or subject using Fluorodeoxyglucose Positron emission tomography.
